(19) United States
(12) Patent Application Publication
(10) Pub. No.: US 2026/0326991 A1
(43) Pub. Date: Oct. 22, 2026

(54) HANGING BLISTERPACK CONNECTOR SCRUBBING DEVICE

(71) Applicant: BECTON, DICKINSON AND COMPANY, Franklin Lakes, NJ (US)

(72) Inventors: Shoshana San Solo, New York, NY (US); Kevin M. Ryan, Whitehouse Station, NJ (US)

(73) Assignee: BECTON, DICKINSON AND COMPANY, Franklin Lakes, NJ (US)

(21) Appl. No.: 19/109,016

(22) PCT Filed: Sep. 21, 2023
(86) PCT No.: PCT/US2023/033386

§ 371 Date: Mar. 5, 2025

Related U.S. Application Data
(60) Provisional application No. 63/408,585, filed on Sep. 21, 2022.

(51) Int. Cl. B65D 75/36; A61L 2/23; A61L 103/15; A61M 39/16; B65D 75/56

(52) U.S. Cl. CPC B65D 75/367; A61L 2/23; A61M 39/16; B65D 75/566; A61L 2103/15

(57) ABSTRACT

A disinfectant strip comprising a plurality of individual blister packs removably connected to one another by perforations, each of the plurality of individual blister packs having a cavity within a first surface of the plurality of individual blister packs. The cavity is sealed by a film and an absorbent medium is disposed within the cavity and sealed by the film, the absorbent medium saturated with a disinfectant fluid or gel. The disinfectant strip is capable of undergoing EtO sterilization as it is made of high barrier material.

### TECHNICAL FIELD

The present disclosure relates the fields of medical disinfection apparatuses and systems, and in particular disinfection caps for uses with IV needleless connectors.

### BACKGROUND

Vascular access devices (VAD's) are commonly used therapeutic devices and include intravenous (IV) catheters. There are two general classifications of VAD's: peripheral catheters and central venous catheters. Bacteria and other microorganisms may gain entry into a patient's vascular system from access hubs and ports/valves upon connection to the VAD when delivering fluids or pharmaceuticals to a patient. Each access hub, connection, port or valve is associated with some risk of transmitting a catheter related bloodstream infection (CRBSI), which can be costly and potentially lethal.

In order to decrease CRBSI cases and to ensure VAD's are used and maintained correctly, standards of practice have been developed, which include disinfecting and cleaning procedures. Disinfection caps have been added to the Society for Healthcare Epidemiology of America (SHEA) guidelines and have also been incorporated into the Infusion Nurses Standards (INS) guidelines.

In developed markets, when utilizing an IV catheter, a needleless connector will typically be used to close off the system and then subsequently accessed to administer medication or other necessary fluids via the catheter to the patient. INS Standards of Practice recommend the use of a needleless connector and state that it should be “consistently and thoroughly disinfected using alcohol, tincture of iodine or chlorhexidine gluconate/alcohol combination prior to each access.” The disinfection of the needleless connector is ultimately intended to aid in the reduction of bacteria that could be living on the surface and possibly lead to a variety of catheter related complications including CRBSI. Nurses will typically utilize a 70% isopropyl alcohol (IPA) pad to complete this disinfection task by doing what is known as “scrubbing the hub.” However, compliance and the level of disinfection attributed to this practice typically relies on the ability and competency of the user. In addition to a lack of compliance to “scrubbing the hub”, it has also been noted through clinician interviews that there is often a variation in scrub time, dry time and the number of times the needleless connector is scrubbed.

Disinfection caps however are commonly injection molded enclosures with the addition of a means of sealing the open end of the injection mold. Furthermore, disinfection caps are commonly individually packaged and individually sterilized during packaging or removably adhered to a strip and packaged together with multiple disinfection caps. These common apparatuses and systems require multiple steps of manufacturing, sterilizing and various methods of injection molding, assembly and packaging.

Thus, there is a need in the art to provide a multi-package disinfection apparatus and system which can provide for low-cost manufacturing, low-complexity assembly and ease of use. There is a specific need to provide a hanging blister pack connector scrubbing device that provides convenient disinfecting units for scrubbing the hub before accessing the line.

### SUMMARY

A first aspect of the present disclosure relates to a disinfectant strip including a body having a first end and a second end defining a length of the body, and a first medial edge and second medial edge, a plurality of individual blister packs connected in series and disposed between the first medial edge and second medial edge of the body. The first end and first medial edge forming a first tab and the second end and second medial edge forming a second tab. Each individual blister pack having a body having a first end and a second end defining a length of the body. Each individual blister pack connected to an adjacent blister pack by a perforation. The first end of the individual blister pack connected to the first medial edge of the first tab, each individual blister pack further having a top surface and a bottom surface, and a pouch extending from the top surface of the individual blister pack with an open end in the bottom surface defining a cavity. The disinfectant strip further includes an absorbent medium disposed within the cavity and sealed by a high-barrier webbing foil. The absorbent medium is impregnated with a disinfectant fluid or gel.

In one or more embodiments, the first tab and second tab include an aperture for hanging the disinfectant strip from a hook. The aperture may have a circular, oval, square, triangular or rectangular shape.

In one or more embodiments, the body of the disinfectant strip further includes side sections connecting the first tab and second tab. The side sections may be connected to side edges of the plurality of individual blister packs.

In one or more embodiments, the body of the disinfectant strip comprises one side section connecting the first tab to the second tab.

In one or more embodiments, the second end of a blister pack is connected to the second medial edge of the second tab.

In one or more embodiments, the high-barrier webbing foil includes weak points or fluid-tight perforations for puncturing the high-barrier webbing foil upon application of lateral forces to one or more wall surfaces of the pouch In one or more embodiments, one or more edges of the high-barrier webbing foil has a pull-tab for peeling the high-barrier webbing foil away from the bottom surface of the individual blister pack.

In one or more embodiments, the high-barrier webbing foil has a thickness of less than 0.012 in.

In one or more embodiments, the high-barrier webbing foil is thermo-formed onto the bottom surface of the individual blister pack.

In one or more embodiments, the absorbent medium is a sponge or disinfectant wipe.

Another aspect of the present disclosure relates to a disinfection device including a plurality of individual blister packs removably connected to one another by perforations. The individual blister packs having a housing including a bottom web formed from a high barrier packaging film with a thickness of less than 0.012 inch. The high barrier packaging film is thermo-formed into a non-circular profile, with no thread features in the interior and no external ribs. In one or more embodiments, the housing has a non-circular profile shape. In one or more specific embodiments, the housing has a polygon shape with corner fillets.

### BRIEF DESCRIPTION OF THE DRAWINGS

FIG. 1 illustrates a top perspective view of a disinfectant strip in accordance with one or more embodiments of the present disclosure;

FIG. 2 illustrates a bottom perspective view of a disinfectant strip in accordance with one or more embodiments of the present disclosure;

FIG. 3 illustrates a top perspective view of a blister pack of a disinfectant strip in accordance with one or more embodiments of the present disclosure; and,

FIG. 4 illustrates a side cross-sectional view of a blister pack of a disinfectant strip in accordance with one or more embodiments of the present disclosure.

FIG. 5 illustrates a top view of a blister pack of a disinfectant strip in accordance with one or more embodiments of the present disclosure.

FIG. 6 illustrates a bottom view of a blister pack of a disinfectant strip in accordance with one or more embodiments of the present disclosure.

FIG. 7 illustrates a left side view of a blister pack of a disinfectant strip in accordance with one or more embodiments of the present disclosure.

FIG. 8 illustrates a right side view of a blister pack of a disinfectant strip in accordance with one or more embodiments of the present disclosure.

FIG. 9 illustrates a front view of a disinfectant strip in accordance with one or more embodiments of the present disclosure.

FIG. 10 illustrates a back side view of a disinfectant strip in accordance with one or more embodiments of the present disclosure.

FIG. 11 illustrates a top view of an embodiment of the disclosure having multiple parallel strips of blister packs of a disinfectant strip in accordance with an alternative embodiment of the present disclosure.

FIG. 12 illustrates a bottom view of an embodiment of the disclosure having multiple parallel strips of blister packs of a disinfectant strip in accordance with an alternative embodiment of the present disclosure.

FIG. 13 illustrates a left side view of an embodiment of the disclosure having multiple parallel strips of blister packs of a disinfectant strip in accordance with an alternative embodiment of the present disclosure.

FIG. 14 illustrates a right side view of an embodiment of the disclosure having multiple parallel strips of blister packs of a disinfectant strip in accordance with an alternative embodiment of the present disclosure.

FIG. 15 illustrates a front view of an embodiment of the disclosure having multiple parallel strips of blister packs of a disinfectant strip in accordance with an alternative embodiment of the present disclosure.

FIG. 16 illustrates back view of an embodiment of the disclosure having multiple parallel strips of blister packs of a disinfectant strip in accordance with an alternative embodiment of the present disclosure.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

For purposes of the description hereinafter, the terms “lateral”, “medial”, “longitudinal”, and derivatives thereof shall relate to the disclosure as it is oriented in the drawing figures. However, it is to be understood that the disclosure may assume alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments disclosed herein are not to be considered as limiting.

As used herein, the use of “a,” “an,” and “the” includes the singular and plural.

As would be readily appreciated by skilled artisans in the relevant art, while descriptive terms such as “thread”, “taper”, “tab”, “wall”, “proximal”, “side”, “distal”, “lateral” and others are used throughout this specification to facilitate understanding, it is not intended to limit any components that can be used in combinations or individually to implement various aspects of the embodiments of the present disclosure.

As used herein, the terms “package” or “packaging” includes any material used to wrap or protect a medical device or product, such as plastic ampoules, drug vials with rubber stoppers, IV solution bags, IV solution pouches and syringes. Packaging can be rigid or flexible. Packaging includes, but is not limited to, medical packaging, pharmaceutical packaging, and child-resistant packaging. Medical and pharmaceutical Packaging can include plastic trays with webbing, blister packs, flow wrap and 3 or 4 sided pouches.

As used herein, the term “microorganism” refers to a microbe or organism that is unicellular or lives in a colony of cellular organisms. Microorganisms are very diverse; they include, but are not limited to bacteria, fungi, archaea, and protozoans.

As used herein, the term “sterilization” refers to a wide variety of techniques employed to attenuate, kill or eliminate harmful or infectious agents. Examples of sterilization procedures include, for example, steam sterilization, ethylene oxide sterilization (EtO sterilization), gas plasma sterilization, ozone sterilization, hydrogen peroxide sterilization, heat sterilization, nitrous dioxide sterilization, or a combination thereof. During manufacturing, pre-filled medical devices (such as drugs or other injectable/infusible solutions which are packaged in gas-permeable containers such as plastic ampoules, drug vials with rubber stoppers, IV solution bags, IV solution pouches and pre-filled flush syringes) are sterilized with non-toxic or non-hazardous sterilization processes such as a steam sterilization in an autoclave. Other medical devices which are less sensitive can undergo subsequent EtO sterilization without any adverse effects to the device sterilized. EtO sterilization can be considered more hazardous to pre-filled medical devices.

As used herein, the term “ethylene oxide (EtO) sterilization” is a common in methods used to prepare a kit of multiple medical devices or accessories for use in a sterile field. Currently, pre-filled saline syringes, plastic ampoules, drug vials with rubber stoppers, IV solution bags, IV solution pouches are packaged in gas permeable packaging which is also permeable to EtO gas. Such devices can be made of glass or high barrier injection molded resin capable of protecting the device itself or the contents within from EtO gas. However, exposure of a plastic ampoules, drug vials with rubber stoppers, IV solution bags, IV solution pouches or pre-filled syringe to EtO gas results in an undesirable effect of increasing the pH of the contents of the plastic ampoules, drug vials with rubber stoppers, IV solution bags, IV solution pouches or pre-filled syringe, e.g. saline. To overcome this undesired effect, medical devices which are resistive to EtO gas are sterilized separately from medical devices which are not. Often, these separate medical devices are later combined in a kit which can then be sterilized as a whole.

The term “high barrier” material as used herein refers to a material which is resistive to EtO gas and can be sterilized with EtO gas (essentially undergoing an entire EtO sterilization). Such materials can include, but are not limited to, high barrier resins, top web films or aluminum alloy films.

The term “disinfectant,” “antiseptic” and “antimicrobial agent” refer to liquids and gels impregnated within an absorbent medium. The absorbent medium can include sponges, foam, or fabric and synthetic wipes (commonly referred to as “alcohol wipes”). The “disinfectant,” “antiseptic” and “antimicrobial agent” can include variations of alcohol or chlorhexidine The “disinfectant,” “antiseptic” and “antimicrobial agent” is selected from the group consisting essentially of isopropyl alcohol, ethanol, 2-propanol, butanol, methylparaben, ethylparaben, propylparaben, propyl gallate, butylated hydroxyanisole (BHA), butylated hydroxytoluene, t-butyl-hydroquinone, chloroxylenol, chlorohexidine, chlorhexidine diacetate, chlorohexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof. The “disinfectant,” “antiseptic” and “antimicrobial agent” can comprise least one of chlorhexidine gluconate and chlorhexidine diacetate.

Embodiments of the present disclosure are directed to a disinfectant device comprising a plurality of individual blister packs removably connected to one another by perforations. Each of the plurality of individual blister packs having a cavity within a first surface of the plurality of individual blister packs. The cavity is sealed by a film and an absorbent medium is disposed within the cavity and sealed by the film, the absorbent medium saturated with a disinfectant fluid or gel. The disinfectant device comprising a plurality of individual blister packs may be in the form of a strip and is capable of undergoing EtO sterilization as it is made of high barrier material. In one or more embodiments, the plurality of individual blister packs are arranged in the form of a strip having a hook eyelet or hanger at the one end to allow the disinfection device to be hung for convenient access to a clinician. In one or more embodiments, the plurality of individual blister packs are arranged in the form of a strip having two apertures or hook eyelets arranged at opposite ends of the strip formed by a plurality of individual blister packs to allow the disinfection device to be hung from either side of the strip for convenient access to a clinician. In one or more embodiments, the disinfection device includes multiple blister pack scrubbing devices that are manufactured together to form a single unitary strip wherein perforations separate the individual blister packs to easily detach an individual or multiple blister packs from the strip. In one or more embodiments, the strips includes up to 10 individual blister packs conjugated together to form a strip for easy hanging on an intravenous (IV) pole. The embodiments of the present disclosure provide the benefits of a low cost alcohol wipe with enhanced durability to allow for stronger mechanical cleaning with an added convenience to ensure clinicians always have the proper materials when needed. One example of the added convenience is that having a strip that may be hung on an IV pole and which includes multiple individual blister pack of alcohol wipes or disinfectant soaked scrubbing devices that are manufactured together to form a single unitary strip wherein perforations separate the individual blister packs allows for easy detachment at each patient's bedside and ensures that clinicians will scrub more often, not having to worry about forgetting supplies. The hanging capability allows the disinfection device to be conveniently located near the practitioner, while preventing direct touch contamination or surface contamination from the patient's bed or person while still affording direct access to practitioner to perform the required sterilization.

An additional benefit of the present disclosure is that having the alcohol wipes or disinfectant soaked scrubbing devices packaged in a blister pack device prolongs both the durability and shelf life of the enclosed alcohol wipes or disinfectant soaked scrubbing devices by preventing degradation from dryness or moisture loss and shielding the contents from temperature fluctuations while maintaining sterility. Due to the hard shell of the blister pack, the foil packaging can be gripped and used as a means for holding the wipes or disinfectant soaked scrubbing devices while scrubbing, thereby minimizing touch contamination of the wipes or disinfectant soaked scrubbing devices. The blister back wipes will be manufactured in individual packages with perforations in between for easy tearing. At one end or both ends of the strip, there will be an aperture, hook eyelet or hole in the strip for easy hanging along an IV pole.

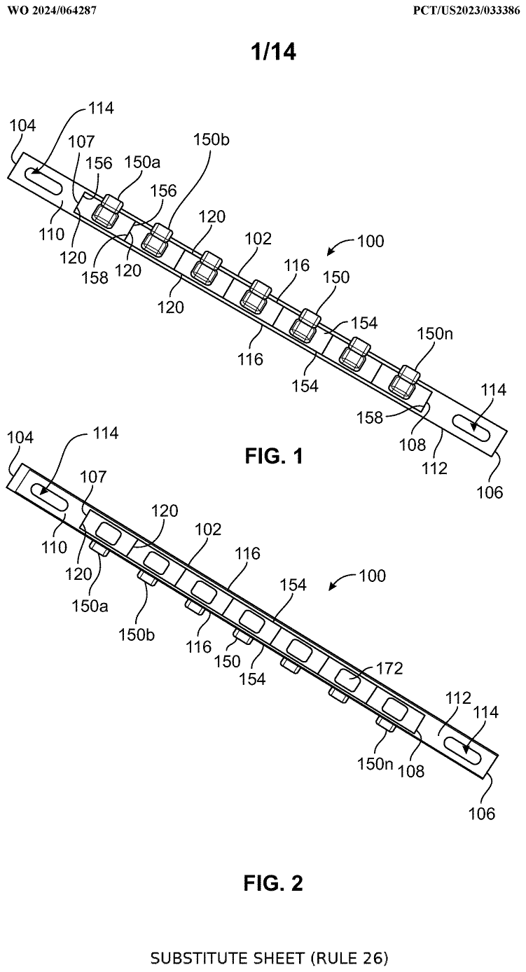

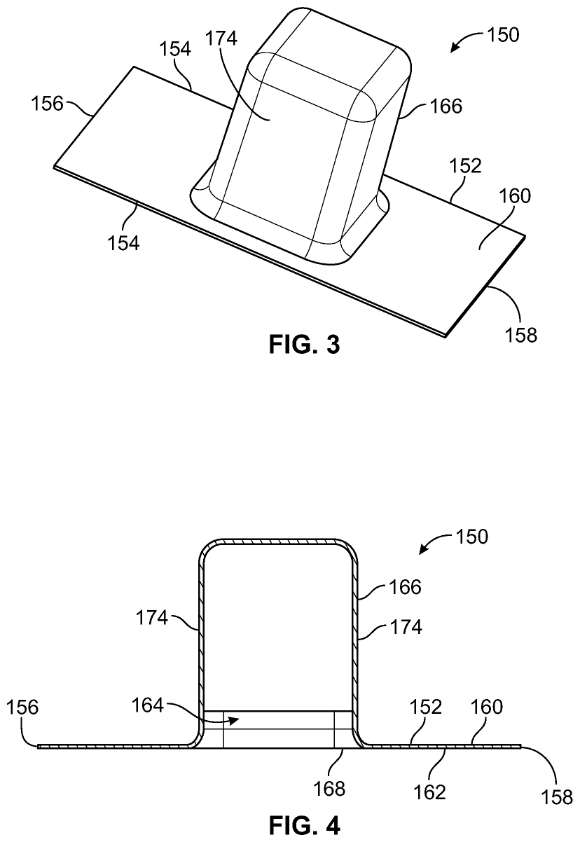

FIG. 1 illustrates a top perspective view and FIG. 2 illustrates a bottom perspective view of a disinfectant strip 100 with a plurality of individual blister packs 150 removably connected to each other by a perforations. FIG. 3 illustrates a top perspective view and FIG. 4 illustrates a side cross-sectional view of an individual blister pack 150.

As shown in FIGS. 1 and 2, the disinfectant strip 100 comprises a rectangular or square body 102 having a first end 104 and a second end 106 defining a length of the body 102. The plurality of individual blister packs 150 are connected in series with one another and are disposed between a first medial edge 107 and second medial edge 108 of the disinfectant strip 100.

The first end 104 and first medial edge 107 form a first tab 110 and the second end 106 and second medial edge 108 form a second tab 112. In some embodiments, the disinfectant strip 100 does not include the second end 106, second medial edge 108 and second tab 112. In some embodiments, the first tab 110 and second tab 112 comprise an aperture 114 for hanging the disinfectant strip 100 from a hook of an IV pole or more generally a hook. In some embodiments, the aperture 114 can have a circular, oval, square, triangular or rectangular shape.

In some embodiments, side sections 116 connect the first tab 110 and second tab 112 and are also connected to side edges 154 of the plurality of individual blister packs 150. In some embodiments, only one side section 116 connects the first tab 110 and second tab 112 and to one side edge 154 of the plurality of individual blister packs 150. Each edge of each individual blister pack 150 is connected to an adjacent edge of an adjacent individual blister pack 150 by perforations 120, and all side edges 154 of the plurality of individual blister packs 150 are connected to the side sections 116 by perforations 120.

As shown in FIGS. 3 and 4, the individual blister pack 150 comprises a rectangular or square body 152 having a first end 156 and a second end 158 defining a length of the body 152. The plurality of individual blister packs 150 are connected in series with one another by a perforation 120 (as shown in FIG. 1) between a second end 158 of a first individual blister pack 150a and the first end 156 of an adjacent (second) individual blister pack 150b (as shown in FIG. 1). The first end 156 of the first individual blister pack 150a is connected to the first medial edge 107 of the first tab 110. Likewise, the second end 158 of a n-blister pack 150n is connected to the second medial edge 108 of the second tab 112. As previously stated, the side edges 154 of the individual blister pack 150 connects to the side sections 116 of the disinfectant strip 100.

The individual blister pack 150 further comprises a top surface 160 and a bottom surface 162 with a pouch 166 extending from the top surface 160 with an open end 168 in the bottom surface 162 defining a cavity 164. The open end as a tapered opening to help facilitate the entry and connection of the needleless connector to be scrubbed. The pouch 166 and cavity 164 can have a square cubical, rectangular cubical or polygonal shape and includes an absorbent medium 170 disposed within. The absorbent medium 170 can be a sponge, foam, or fabric and synthetic wipes (commonly referred to as “alcohol wipes”) and is impregnated with a disinfectant. The cavity 164 is sealed by a high-barrier webbing foil 172 (as best shown in FIG. 2). The high-barrier webbing foil 172 can include weak points or fluid-tight perforations for puncturing the high-barrier webbing foil 172 upon application of lateral forces to one or more wall surfaces 174 of the pouch 166. In some embodiments, one or more edges of the high-barrier webbing foil 172 has a pull-tab for peeling the high-barrier webbing foil 172 away from the bottom surface 162 of the individual blister pack 150. In some embodiments, the high-barrier webbing foil 172 can have a thickness of less than 0.012 in. In some embodiments, the high-barrier webbing foil 172 is thermo-formed onto the bottom surface 162 of the individual blister pack 150.

In operation, the disinfectant strip 100 can be hung from an IV line or more generally a hook by the aperture 114 of either the first tab 110 or second tab 112. A user or practitioner can tear an individual blister pack 150 while the plurality of individual blister packs still hang from the disinfectant strip 100. The user can then squeeze one or more wall surfaces 174 until the high-barrier webbing foil 172 tears, at which point the user can access the absorbent medium 170 disposed within. Alternatively, the user can tear the high-barrier webbing foil 172 off the bottom surface bottom surface 162 of the individual blister pack 150 by pulling on the pull tab.

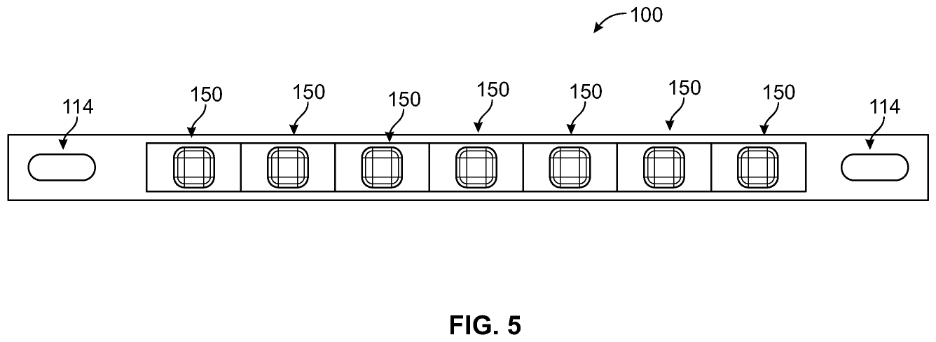

FIG. 5 illustrates a top view of the disinfectant strip 100 of FIG. 1 with a plurality of individual blister packs 150 removably connected to each other by a perforations in accordance with one or more embodiments of the present disclosure.

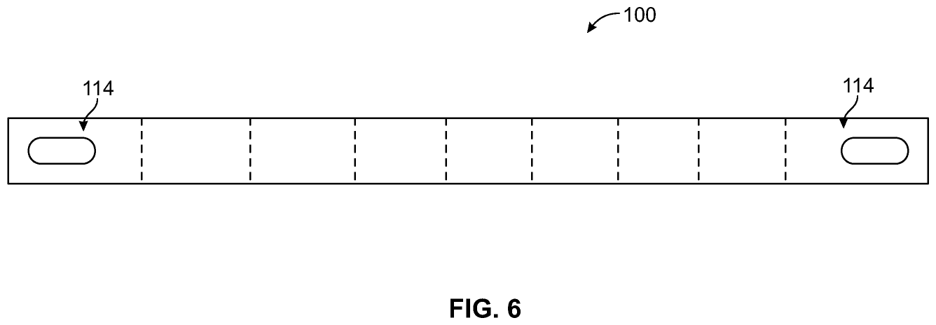

FIG. 6 illustrates a bottom view of the disinfectant strip 100 of FIG. 1 with a plurality of individual blister packs 150 removably connected to each other by a perforations in accordance with one or more embodiments of the present disclosure.

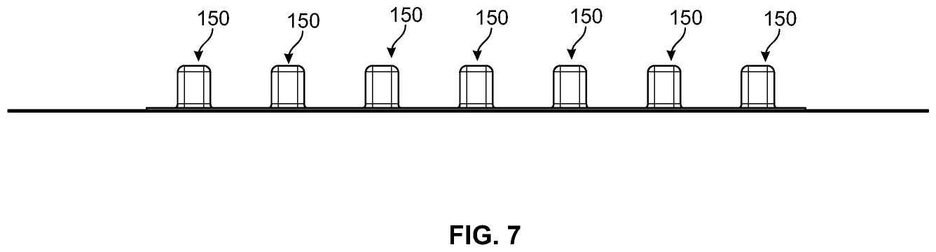

FIG. 7 illustrates a left side view of a disinfectant strip 100 of FIG. 1 with a plurality of individual blister packs 150 in accordance with one or more embodiments of the present disclosure.

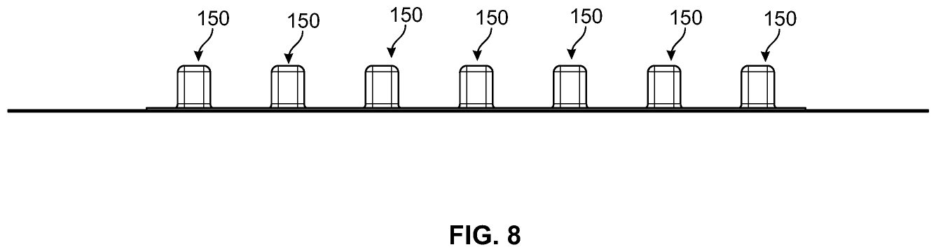

FIG. 8 illustrates a right side view of a disinfectant strip 100 of FIG. 1 with a plurality of individual blister packs 150 in accordance with one or more embodiments of the present disclosure.

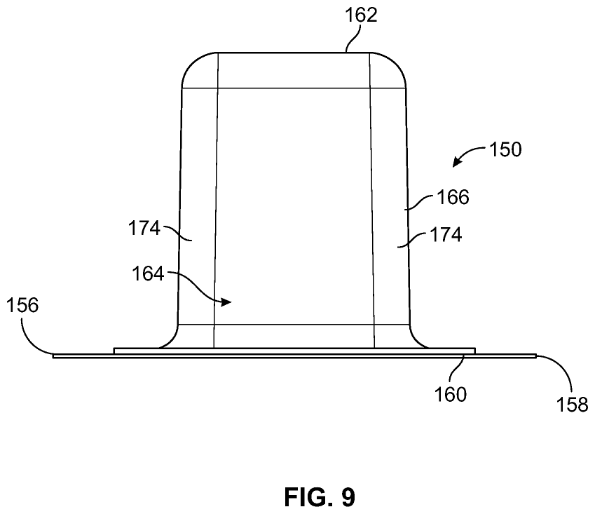

FIG. 9 illustrates a front view of a disinfectant strip 100 of FIG. 1 in accordance with one or more embodiments of the present disclosure.

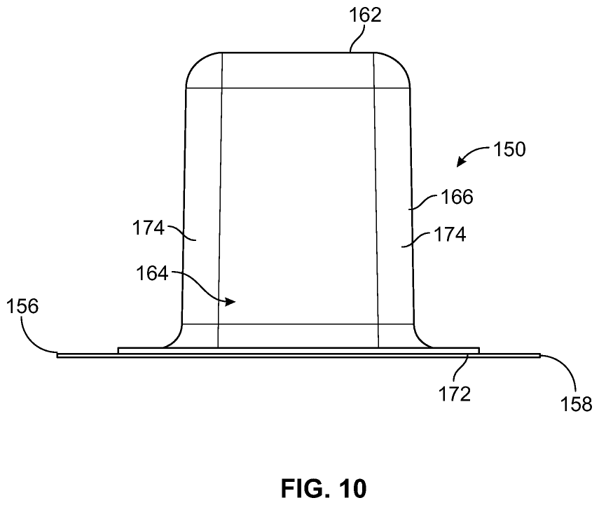

FIG. 10 illustrates a back side view of a disinfectant strip 100 of FIG. 1 in accordance with one or more embodiments of the present disclosure.

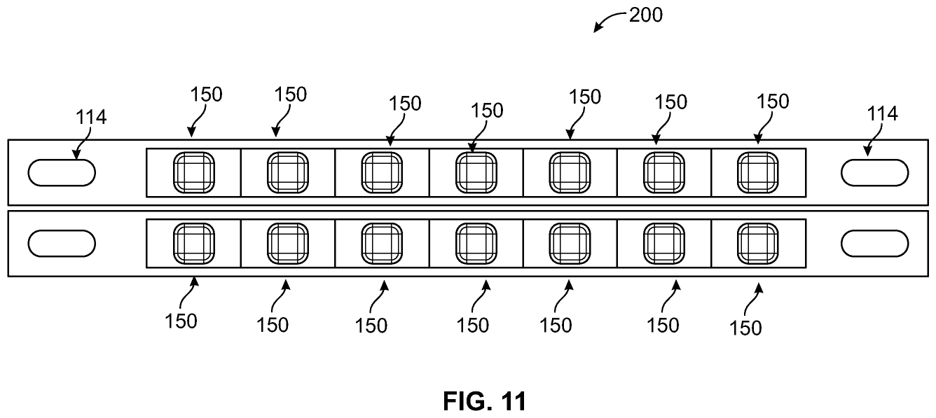

An alternative embodiment of the present disclosure is shown in FIG. 11. FIG. 11 illustrates a top view of an embodiment of the disclosure having a unit comprising multiple parallel strips of disinfectant strips 100 with a plurality of individual blister packs 150 removably connected to each other by a perforations. The multiple parallel strips of disinfectant may be removably connected to each other by one or more perforations. It is envisioned that up to 10 multiple parallel strips may be removably connected to each other by a perforations.

As shown in FIGS. 11-16, the disinfectant strip 200 comprises two or more disinfectant strips 100 arranged in a parallel orientation. The two or more disinfectant strips 100 arranged in a parallel orientation may be connected via perforation or adhesive to allow for easy separation as the individual blister packs 150 are used by the practitioner. In one or more embodiments, similar to those shown in FIG. 1, have a rectangular or square body 102 having a first end 104 and a second end 106 defining a length of the body 102. The plurality of individual blister packs 150 are connected in series with one another and are disposed between a first medial edge 107 and second medial edge 108 of the disinfectant strip 100.

The first end 104 and first medial edge 107 form a first tab 110 and the second end 106 and second medial edge 108 form a second tab 112. In some embodiments, the disinfectant strip 100 does not include the second end 106, second medial edge 108 and second tab 112. In some embodiments, the first tab 110 and second tab 112 comprise an aperture 114 for hanging the disinfectant strip 100 from a hook of an IV pole or more generally a hook. In some embodiments, the aperture 114 can have a circular, oval, square, triangular or rectangular shape.

In some embodiments, side sections 116 connect the first tab 110 and second tab 112 and are also connected to side edges 154 of the plurality of individual blister packs 150. In some embodiments, only one side section 116 connects the first tab 110 and second tab 112 and to one side edge 154 of the plurality of individual blister packs 150. Each edge of each individual blister pack 150 is connected to an adjacent edge of an adjacent individual blister pack 150 by perforations 120, and all side edges 154 of the plurality of individual blister packs 150 are connected to the side sections 116 by perforations 120.

As shown in FIGS. 3 and 4, the individual blister pack 150 comprises a rectangular or square body 152 having a first end 156 and a second end 158 defining a length of the body 152. The plurality of individual blister packs 150 are connected in series with one another by a perforation 120 (as shown in FIG. 1) between a second end 158 of a first individual blister pack 150a and the first end 156 of an adjacent (second) individual blister pack 150b (as shown in FIG. 1). The first end 156 of the first individual blister pack 150a is connected to the first medial edge 107 of the first tab 110. Likewise, the second end 158 of a n-blister pack 150n is connected to the second medial edge 108 of the second tab 112. As previously stated, the side edges 154 of the individual blister pack 150 connects to the side sections 116 of the disinfectant strip 100.

The individual blister pack 150 further comprises a top surface 160 and a bottom surface 162 with a pouch 166 extending from the top surface 160 with an open end 168 in the bottom surface 162 defining a cavity 164. The pouch 166 and cavity 164 can have a square cubical, rectangular cubical or polygonal shape and includes an absorbent medium 170 disposed within. The absorbent medium 170 can be a sponge, foam, or fabric and synthetic wipes (commonly referred to as “alcohol wipes”) and is impregnated with a disinfectant. The cavity 164 is sealed by a high-barrier webbing foil 172 (as best shown in FIG. 2). The high-barrier webbing foil 172 can include weak points or fluid-tight perforations for puncturing the high-barrier webbing foil 172 upon application of lateral forces to one or more wall surfaces 174 of the pouch 166. In some embodiments, one or more edges of the high-barrier webbing foil 172 has a pull-tab for peeling the high-barrier webbing foil 172 away from the bottom surface 162 of the individual blister pack 150. In some embodiments, the high-barrier webbing foil 172 can have a thickness of less than 0.012 in. In some embodiments, the high-barrier webbing foil 172 is thermo-formed onto the bottom surface 162 of the individual blister pack 150. In operation, the disinfectant strip 200 can be hung from an IV line or more generally a hook by the aperture 114 of either the first tab 110 or second tab 112. A user or practitioner can tear an individual blister pack 150 while the plurality of individual blister packs still hang from the disinfectant strip 100. The user can then squeeze one or more wall surfaces 174 until the high-barrier webbing foil 172 tears, at which point the user can access the absorbent medium 170 disposed within. Alternatively, the user can tear the high-barrier webbing foil 172 off the bottom surface bottom surface 162 of the individual blister pack 150 by pulling on the pull tab.

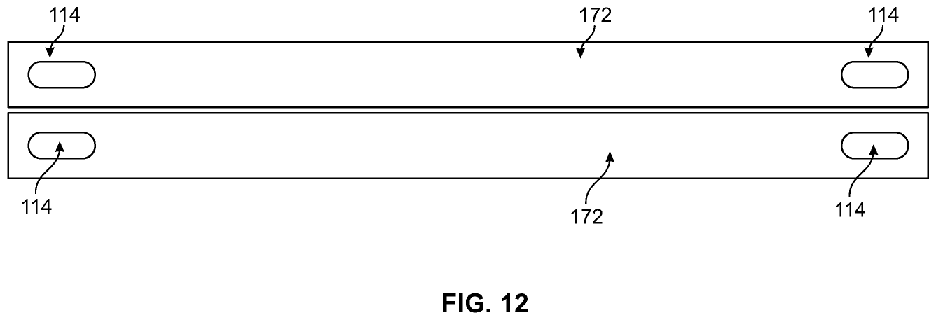

FIG. 12 illustrates a bottom view of the alternative embodiment shown in FIG. 11 having multiple parallel strips of blister packs of a disinfectant strip in accordance with an alternative embodiment of the present disclosure.

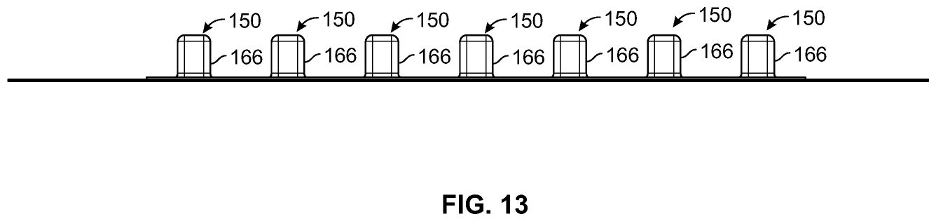

FIG. 13 illustrates a left side view of the alternative embodiment shown in FIG. 11 having multiple parallel strips of blister packs of a disinfectant strip in accordance with an alternative embodiment of the present disclosure.

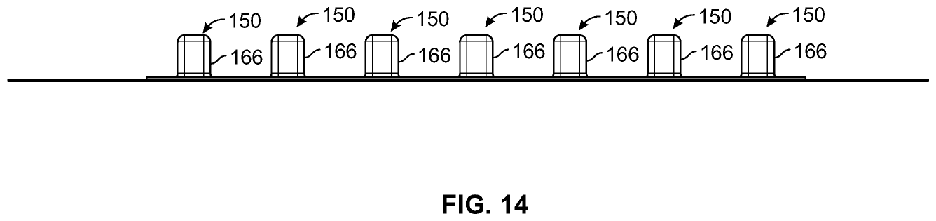

FIG. 14 illustrates a right side view of the alternative embodiment shown in FIG. 11 having multiple parallel strips of blister packs 150 of a disinfectant strip in accordance with an alternative embodiment of the present disclosure.

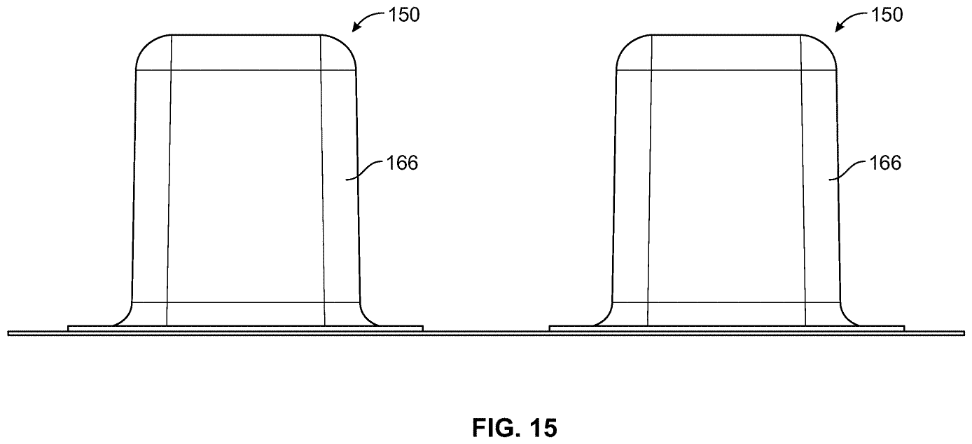

FIG. 15 illustrates a front view of the alternative embodiment shown in FIG. 11 having multiple parallel strips of blister packs of a disinfectant strip in accordance with an alternative embodiment of the present disclosure.

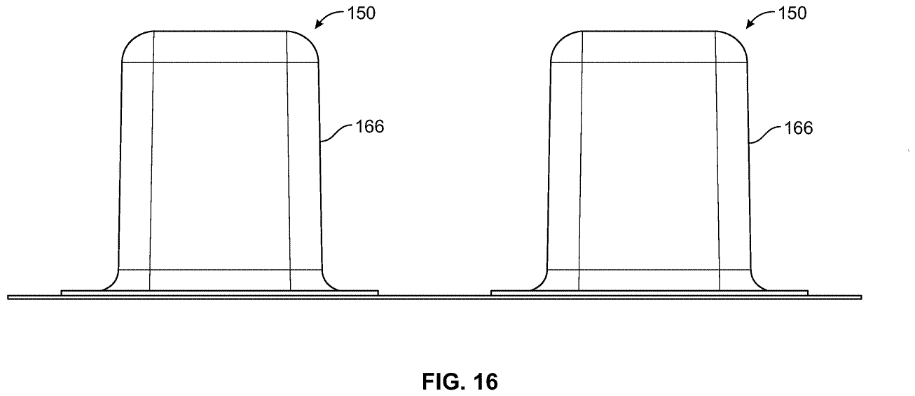

FIG. 16 illustrates a back view of the alternative embodiment shown in FIG. 11 having multiple parallel strips of blister packs of a disinfectant strip in accordance with an alternative embodiment of the present disclosure.

In some embodiments, components of the disinfectant strip 100 are made from materials which are resistant to EtO gas and thus able to undergo EtO sterilization. In particular, in some embodiments, the body 102 of the disinfectant strip 100 and the high-barrier webbing foil 172 are made from foils or plastics resistant to EtO gas.

Due to components of the disinfectant strip 100 being resistant to EtO gas, and thus able to undergo EtO sterilization, the disinfectant strip 100 and the plurality of individual blister packs 150 can be manufactured and sealed as a package of multiple blister packs 150, and subsequently the entire package can undergo EtO sterilization.

The disinfection strip of the present disclosure also provides a benefit in that the Cost Of Goods Sold (COGS) is less than disinfection caps currently on the market due to the materials and manufacturing process of producing the bottom web packaging of the housing.

In one or more embodiments, the disinfection device comprises a plurality of individual blister packs removably connected to one another by perforations, the individual blister packs having a housing including a bottom web formed from a high barrier packaging film with a thickness of less than 0.012 inch, wherein the high barrier packaging film is thermo-formed into a non-circular profile, with no thread features in the interior and no external ribs. In one or more embodiments, the housing has a non-circular profile shape. In one or more embodiments, the housing is a polygon with corner fillets.

The disinfection strip of the present disclosure includes perforated disinfecting units which are convenient to use and therefore increased compliance from clinicians. There is a present need of clinicians a convenient disinfection device to reduce instances in which the clinician forgets to bring all the disinfection supplies need and therefore resulting in performing a disinfection procedure without all of the necessary supplies or alternatively dropping one of the required disinfection supplies while performing a disinfection procedure and completing the procedure without getting a new replacement disinfection supply to complete the disinfection procedure. The disinfection strip of the present disclosure seeks to address the aforementioned deficiencies of the disinfection supplies currently available to the clinician.

Another aspect of the packaging and disinfection strip of the present disclosure is to provide a perforated disinfection strip that is firm enough to be held as an individual disinfection device once an individual disinfection unit is removed from the perforated strip. Thus, the disinfection device of the present disclosure So while it is a cost savings project, it can also be viewed as a cost-effective alternate option that maintains sterility in the field. Clinicians don't need to use their (likely not perfectly clean gloved) hands to scrub but can use the packaging, and possibly the polygon shape to grip.

In one or more embodiment, the individual disinfection unit is a polygon shape which allows for better gripping, almost acting as non-slip configuration. The different sides of the polygon shape allow for those with different sized hands to grip in the most ergonomic way. The sides of the polygon shape also allow for more force to be delivered in the scrubbing.

### EMBODIMENTS

Various embodiments are listed below. It will be understood that the embodiments listed below may be combined with all aspects and other embodiments in accordance with the scope of the invention.
Embodiment (a). A disinfectant strip comprising:
a body having a first end and a second end defining a length of the body, and a first medial edge and second medial edge, the first end and first medial edge forming a first tab and the second end and second medial edge forming a second tab;
a plurality of individual blister packs connected in series and disposed between the first medial edge and second medial edge of the body, each individual blister pack having a body having a first end and a second end defining a length of the body, each individual blister pack connected to an adjacent blister pack by a perforation, and the first end of the individual blister pack connected to the first medial edge of the first tab, each individual blister pack further having a top surface and a bottom surface, and a pouch extending from the top surface of the individual blister pack with an open end in the bottom surface defining a cavity; and,
an absorbent medium disposed within the cavity and sealed by a high-barrier webbing foil, the absorbent medium impregnated with a disinfectant fluid or gel.
Embodiment (b). The disinfectant strip of embodiment (a), wherein the first tab and second tab comprise an aperture for hanging the disinfectant strip from a hook.
Embodiment (c). The disinfectant strip of embodiment (b), wherein the aperture has a circular, oval, square, triangular or rectangular shape.
Embodiment (d). The disinfectant strip of embodiment (a), wherein the body of the disinfectant strip further comprises side sections connecting the first tab and second tab.
Embodiment (e). The disinfectant strip of embodiment (d), wherein the side sections are connected to side edges of the plurality of individual blister packs.
Embodiment (f). The disinfectant strip of embodiment (a), wherein the body of the disinfectant strip comprises one side section connecting the first tab to the second tab.
Embodiment (g). The disinfectant strip of embodiment (a), wherein the second end of the blister pack is connected to the second medial edge of the second tab.
Embodiment (h). The disinfectant strip of embodiment (a), wherein the high-barrier webbing foil include weak points or fluid-tight perforations for puncturing the high-barrier webbing foil upon application of lateral forces to one or more wall surfaces of the pouch Embodiment (i). The disinfectant strip of embodiment (a), wherein one or more edges of the high-barrier webbing foil has a pull-tab for peeling the high-barrier webbing foil away from the bottom surface of the individual blister pack.
Embodiment (j). The disinfectant strip of embodiment (a), wherein the high-barrier webbing foil has a thickness of less than 0.012 in.
Embodiment (k). The disinfectant strip of embodiment (a), wherein the high-barrier webbing foil is thermo-formed onto the bottom surface of the individual blister pack.
Embodiment (l). The disinfectant strip of embodiment (a), wherein the absorbent medium is a sponge.
Embodiment (m). The disinfectant strip of embodiment (a), wherein the absorbent medium is a disinfectant wipe.
Embodiment (n). A disinfection device comprising: a plurality of individual blister packs removably connected to one another by perforations, the individual blister packs having a housing including a bottom web formed from a high barrier packaging film with a thickness of less than 0.012 inch, wherein the high barrier packaging film is thermo-formed into a non-circular profile, with no thread features in the interior and no external ribs.
Embodiment (o). The disinfection device of embodiment (n), wherein the housing has a non-circular profile shape.
Embodiment (p). The disinfection device of embodiment (o), wherein the housing has a polygon shape with corner fillets.
Embodiment (q). A disinfectant strip comprising:
two or more bodies having a first end and a second end defining a length of the body, and a first medial edge and second medial edge, the first end and first medial edge forming a first tab and the second end and second medial edge forming a second tab;
a plurality of individual blister packs connected in series and disposed between the first medial edge and second medial edge of the body, each individual blister pack having a body having a first end and a second end defining a length of the body, each individual blister pack connected to an adjacent blister pack by a perforation, and the first end of the individual blister pack connected to the first medial edge of the first tab, each individual blister pack further having a top surface and a bottom surface, and a pouch extending from the top surface of the individual blister pack with an open end in the bottom surface defining a cavity; and,
an absorbent medium disposed within the cavity and sealed by a high-barrier webbing foil, the absorbent medium impregnated with a disinfectant fluid or gel the two or more bodies connected in parallel orientation by a perforated connection.
Embodiment (r). The disinfectant strip of embodiment (q), wherein the first tab and second tab comprise an aperture for hanging the disinfectant strip from a hook.
Embodiment(s). The disinfectant strip of embodiment (r), wherein the aperture has a circular, oval, square, triangular or rectangular shape.
Embodiment (t). The disinfectant strip of embodiment (q), wherein the body of the disinfectant strip further comprises side sections connecting the first tab and second tab.
Embodiment (u). The disinfectant strip of embodiment (t), wherein the side sections are connected to side edges of the plurality of individual blister packs.
Embodiment (v). The disinfectant strip of embodiment (q), wherein the body of the disinfectant strip comprises one side section connecting the first tab to the second tab.
Embodiment (w). The disinfectant strip of embodiment (q), wherein the second end of the blister pack is connected to the second medial edge of the second tab.
Embodiment (x). The disinfectant strip of embodiment (q), wherein the high-barrier webbing foil include weak points or fluid-tight perforations for puncturing the high-barrier webbing foil upon application of lateral forces to one or more wall surfaces of the pouch.
Embodiment (y). The disinfectant strip of embodiment (q), wherein one or more edges of the high-barrier webbing foil has a pull-tab for peeling the high-barrier webbing foil away from the bottom surface of the individual blister pack.
Embodiment (z). The disinfectant strip of embodiment (q), wherein the high-barrier webbing foil has a thickness of less than 0.012 in.
Embodiment (aa). The disinfectant strip of embodiment (q), wherein the high-barrier webbing foil is thermo-formed onto the bottom surface of the individual blister pack.
Embodiment (bb). The disinfectant strip of embodiment (q), wherein the absorbent medium is a sponge.
Embodiment (cc). The disinfectant strip of embodiment (q), wherein the absorbent medium is a disinfectant wipe.

While the present disclosure has been shown and described with reference to certain exemplary embodiments thereof, it will be understood by those skilled in the art that various changes in form and details may be made therein without departing from the spirit and scope of the embodiments of the present disclosure. Furthermore, any of the features or elements of any exemplary implementations of the embodiments of the present disclosure as described above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of invention.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has provided a description with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

1. A disinfectant strip comprising:
a body having a first end and a second end defining a length of the body, and a first medial edge and second medial edge, the first end and first medial edge forming a first tab and the second end and second medial edge forming a second tab, wherein the body of the disinfectant strip comprises one side section connecting the first tab to the second tab;
a plurality of individual blister packs connected in series and disposed between the first medial edge and second medial edge of the body, each individual blister pack having a body having a first end and a second end defining a length of the body, each individual blister pack connected to an adjacent blister pack by a perforation, and the first end of the individual blister pack connected to the first medial edge of the first tab, each individual blister pack further having a top surface and a bottom surface, and a pouch extending from the top surface of the individual blister pack with an open end in the bottom surface defining a cavity; and,
an absorbent medium disposed within the cavity and sealed by a high-barrier webbing foil, the absorbent medium impregnated with a disinfectant fluid or gel.

2. The disinfectant strip of claim 1, wherein the first tab and second tab comprise an aperture for hanging the disinfectant strip from a hook.

3. The disinfectant strip of claim 2, wherein the aperture has a circular, oval, square, triangular or rectangular shape.

4. The disinfectant strip of claim 1, wherein the body of the disinfectant strip further comprises side sections connecting the first tab and second tab.

5. The disinfectant strip of claim 4, wherein the side sections are connected to side edges of the plurality of individual blister packs.

6. (canceled)

7. The disinfectant strip of claim 1, wherein the second end of the blister pack is connected to the second medial edge of the second tab.

8. The disinfectant strip of claim 1, wherein the high-barrier webbing foil include weak points or fluid-tight perforations for puncturing the high-barrier webbing foil upon application of lateral forces to one or more wall surfaces of the pouch

9. The disinfectant strip of claim 1, wherein one or more edges of the high-barrier webbing foil has a pull-tab for peeling the high-barrier webbing foil away from the bottom surface of the individual blister pack.

10. The disinfectant strip of claim 1, wherein the high-barrier webbing foil has a thickness of less than 0.012 in.

11. The disinfectant strip of claim 1, wherein the high-barrier webbing foil is thermo-formed onto the bottom surface of the individual blister pack.

12. The disinfectant strip of claim 1, wherein the absorbent medium is a sponge.

13. The disinfectant strip of claim 1, wherein the absorbent medium is a disinfectant wipe.

14. A disinfection device comprising: a plurality of individual blister packs removably connected to one another by perforations, the individual blister packs having a housing including a bottom web formed from a high barrier packaging film with a thickness of less than 0.012 inch, wherein the high barrier packaging film is thermo-formed into a non-circular profile, with no thread features in the interior and no external ribs.

15. The disinfection device of claim 14, wherein the housing has a non-circular profile shape.

16. The disinfection device of claim 15, wherein the housing has a polygon shape with corner fillets.

17. A disinfectant strip comprising:
two or more bodies having a first end and a second end defining a length of the body, and a first medial edge and second medial edge, the first end and first medial edge forming a first tab and the second end and second medial edge forming a second tab;
a plurality of individual blister packs connected in series and disposed between the first medial edge and second medial edge of the body, each individual blister pack having a body having a first end and a second end defining a length of the body, each individual blister pack connected to an adjacent blister pack by a perforation, and the first end of the individual blister pack connected to the first medial edge of the first tab, each individual blister pack further having a top surface and a bottom surface, and a pouch extending from the top surface of the individual blister pack with an open end in the bottom surface defining a cavity; and,
an absorbent medium disposed within the cavity and sealed by a high-barrier webbing foil, the absorbent medium impregnated with a disinfectant fluid or gel
the two or more bodies connected in parallel orientation by a perforated connection.

18. The disinfectant strip of claim 17, wherein the first tab and second tab comprise an aperture for hanging the disinfectant strip from a hook.

19. The disinfectant strip of claim 18, wherein the aperture has a circular, oval, square, triangular or rectangular shape.

20. The disinfectant strip of claim 17, wherein the body of the disinfectant strip further comprises side sections connecting the first tab and second tab.

21. The disinfectant strip of claim 20, wherein the side sections are connected to side edges of the plurality of individual blister packs.

22. The disinfectant strip of claim 17, wherein the body of the disinfectant strip comprises one side section connecting the first tab to the second tab.

23. The disinfectant strip of claim 17, wherein the second end of the blister pack is connected to the second medial edge of the second tab.

24. The disinfectant strip of claim 17, wherein the high-barrier webbing foil include weak points or fluid-tight perforations for puncturing the high-barrier webbing foil upon application of lateral forces to one or more wall surfaces of the pouch

25. The disinfectant strip of claim 17, wherein one or more edges of the high-barrier webbing foil has a pull-tab for peeling the high-barrier webbing foil away from the bottom surface of the individual blister pack.

26. The disinfectant strip of claim 17, wherein the high-barrier webbing foil has a thickness of less than 0.012 in.

27. The disinfectant strip of claim 17, wherein the high-barrier webbing foil is thermo-formed onto the bottom surface of the individual blister pack.

28. The disinfectant strip of claim 17, wherein the absorbent medium is a sponge.

29. The disinfectant strip of claim 17, wherein the absorbent medium is a disinfectant wipe.
